(19) United States
(12) Patent Application Publication
(10) Pub. No.: US 2016/0000654 A1
(43) Pub. Date: Jan. 7, 2016

(54) METHOD FOR DETERMINING FORMULATION ORIENTATION OF ASYMMETRIC MULTI-LAYERED OSMOTIC TABLETS

(71) Applicant: CADILA HEALTHCARE LIMITED, Ahmedabad (IN)

(72) Inventors: Manish CHAWLA, Ahmedabad (IN); Shailesh Vishwanath BIRADAR, Ahmedabad (IN); Mahendra PATEL, Milltown, NJ (US)

(73) Assignee: CADILA HEALTHCARE LIMITED, Ahmedabad (IN)

(21) Appl. No.: 14/791,900

(22) Filed: Jul. 6, 2015

(30) Foreign Application Priority Data
Jul. 7, 2014 (IN) 2210/MUM/2014

(51) Int. Cl. A61J 3/10; A61K 31/519; A61K 9/20; A61K 31/4458

(52) U.S. Cl. CPC A61J 3/10; A61K 31/4458; A61K 31/519; A61K 9/2086; A61K 9/2095

(57) ABSTRACT

The present invention relates to a method for preparing asymmetric multi-layered osmotic tablets using rapid and accurate determination of the orientation of tablets with respect to different internal formulation layers proximate to the opposite curved surfaces. In particular, the invention relates to detect degree of curvature using an image recognition system and drill the desired surface using a laser beam.

### FIELD OF THE INVENTION

The present invention relates to a method for preparing asymmetric multi-layered osmotic tablets using rapid and accurate determination of the orientation of tablets with respect to different internal formulation layers proximate to the opposite curved surfaces. In particular, the invention relates to detect degree of curvature using an image recognition system and drill the desired surface using a laser beam.

### BACKGROUND OF THE INVENTION

Osmotic preparations are typical representatives of sustained and controlled release preparations, characterized in that they use osmotic pressure as the driving force for drug release and follow the zero-order release kinetics. Osmotic preparations have become a hot topic of research and development all over the world. Among them, osmotic tablet is the most common dosage form of oral osmotic controlled release preparations.

Based on the structural characteristics, oral osmotic preparations can be divided into two types: mono-compartment osmotic pumps and multi-compartment osmotic pumps. The mono-compartment osmotic pumps are generally used for water-soluble drugs, and consist of a tablet core and a coating film. The tablet core consists of a drug and a high-permeability material. The coating film is commonly a rigid semipermeable membrane formed by a polymer material such as cellulose acetate or ethyl cellulose, and one or more releasing orifice(s) are usually drilled by laser or other means (such as mechanical force) on the semipermeable membrane and used as the output channel of drugs. When being used, the high-permeability material in the tablet core produces high osmotic pressure after being dissolved, then a static pressure difference between inside and outside of the semipermeable membrane is formed. Thus, under this pressure difference, a drug suspension or solution outflows from the tablet, while external water inflows into the tablet, moreover, the inflowing speed of water is equal to the outflowing speed of the drug suspension or solution. Mono-compartment osmotic pump controlled release tablets are mainly suitable for water-soluble drugs, and not applicable to water-insoluble drugs, especially poorly water-insoluble drugs. In addition, due to the limitation of structure, mono-compartment osmotic pumps will no longer release drugs at a constant rate at the late stage of release, and the osmotic pressures are reduced and may even cause drug residue in preparations, like ordinary sustained-release formulations. Due to the above-mentioned problems present in mono-compartment osmotic pumps, multi-compartment osmotic pumps have subsequently been developed.

Multi-compartment osmotic pumps consist of at least two layers: a drug-containing layer and a push layer, which constitute a drug compartment and a force compartment respectively. The most widely used osmotic pumps are double-compartment osmotic pumps. The drug-containing layer consists of a drug, a penetration-promoting agent and a suspending agent. The push layer as consists of one or more swellable polymer materials, an osmogen and a penetration-promoting agent. When being used, water enters into the tablet core through the semipermeable membrane, resulting in softening of the drug-containing layer by absorbing water, meanwhile, the polymer material in the push layer swells by absorbing water and squeeze the drug compartment, so that drug is released from the releasing orifice. A constant osmotic pressure will keep a constant speed of water entering into the tablet core, thereby keep a constant swelling speed of the polymer material by absorbing water in order to maintain the persistence of a constant osmotic pressure and achieve a constant drug release rate. In addition, the drug, whether existing in a solution or a suspension, can be squeezed out of the semipermeable membrane by the swollen push layer. However, mono-compartment osmotic pump controlled release tablets will cause the penetration-promoting agent and the drug being separated from each other during the delivery of poorly water-soluble drugs, thereby resulting in drug residue in the tablet core. Thus, multi-compartment osmotic pumps are applicable to all types of drugs, and they have more obvious advantages in the aspect of delivering poorly water-soluble drugs compared with mono-compartment osmotic pump controlled release tablets.

Colorants may be used as an indicator of different formulation layers in multi-layered osmotic dosage forms. Formulating different formulation layers with different colorants is a useful quality control method that helps ensure that the different formulation layers are distinguishable from each other during the manufacturing process. Different colors included for different layers to determine the formulation orientation of the dosage form for a particular processing involved in manufacturing of multi-layered osmotic dosage forms. An example of such a processing step is drilling of a delivery orifice in a multi-layered osmotic dosage form. These dosage forms have an internal compartment containing at least one drug-containing layer, at least one expandable polymer-push layer and, optionally, one or more drug-free layers to produce a desired release pattern such as delayed or zero order release. The internal compartment is surrounded by a membrane that is at least partially semipermeable and at least one delivery port is formed through the membrane at an appropriate location to permit release of drug-containing formulation from within the compartment. The expandable polymer-containing layer is known as a “push” layer because, following oral administration, fluid is imbibed through the semipermeable membrane causing the drug-containing layer(s) and any optional drug-free layer(s) to form a dispensable formulation and causing the polymer layer to expand and “push” the dispensable formulation through the drilled port.

Such osmotic dosage forms are typically manufactured by compressing the component dispensable formulation-forming layer(s) and the push layer(s) together to form a multi-layered internal core, applying the semipermeable membrane around the core and then drilling, typically with a laser, an appropriate delivery port. Generally, the push layer is adjacent to one end, the “push end,” of the tablet and the opposite end is the “dispensing end” that is proximal to the dispensable formulation-forming layer(s) within the dosage form. Proper operation of the dosage form requires that the delivery port be formed in the dispensing end of the dosage form and not in the push end of the dosage form. Thus, at some point prior to the laser drilling step, multi-layered osmotic tablet should be oriented by some mechanical means such that laser drilling is done in the dispensing end and not in push end. To have precise laser drilling at desired dispensing end, colors are added to at least one layer, generally in the drug(s) layer of multi-layered osmotic tablet. Then, a contrast or color detector, which is placed just before laser gun, detect the orientation of colored surface and convey signal to laser gun regarding the orientation of colored layer, to be passed through laser gun. Based on location of color in particular layer of multi-layered osmotic tablet, laser will be fired either on colored surface layer. Twin laser gun system is currently used, which ensure laser drilling on colored surface. The top located laser gun will fire only when colored layer is oriented upward and the bottom located laser gun will fire only when colored surface is oriented downward.

Useful methods and apparatus for determining the formulation orientation of such tablets and for drilling the delivery ports in the dispensing ends of the tablets are disclosed and claimed in U.S. Pat. Nos. 5,294,770 and 5,399,828 owned by Alza Corporation. In accord with these inventions, multi-layered osmotic tablets are supplied in a manner that permits laser access to both the front and the back as surface of the dosage form. A suitable color detector is used to determine which surface encompasses the dispensing end of the tablet and, then, a laser controller directs the laser to drill at least one delivery port in that end.

The above-described methods have been shown to be especially satisfactory for conventional tablet shapes where the dispensing end and the push end of the tablet coincide with the front and back surfaces of the tablet. Because these surfaces are relatively broad and flat, a color detector is able to accurately and rapidly determine the color and generate an appropriate signal to direct the laser. More recently, it has been discovered that capsule-shaped osmotic tablets having the dispensing end at one narrow and rounded end of the capsule-shaped tablet and the push end is at the opposite narrow and rounded end of the capsule-shaped tablet are preferable to conventional tablet shapes for certain applications. Unfortunately, because the narrow and rounded ends of the capsule-shaped tablets scatter a significant portion of light directed thereon, the above-described methods for determining the formulation orientation of the dosage forms by detecting the color at the narrow and rounded ends corresponding to the dispensing end and push end of the tablet are not satisfactory.

U.S. Pat. No. 7,521,067 discloses method for determining orientation of tri-layered osmotic capsule shaped tablet by applying dark and light colors to different layers of the tablet, However, in this case only those tablets were subjected to laser drilling which were properly oriented, remaining were removed by the rectifier.

Pharmaceutical manufacturing in general requires high speed, efficiency and accuracy and it is generally desirable to provide as many automated steps as possible. It would be an advance in the art to develop rapid and accurate automated detection methods and apparatus for determining the formulation orientation of multi-layered capsule-shaped osmotic dosage forms with respect to different internal formulation layers proximate to the opposite narrow and rounded ends of the tablets.

Hence, there still remains a need for an alternative method for rapid and accurate determination of the orientation of tablets with respect to different internal formulation layers proximate to the opposite curved surfaces and drilling the desired surface.

### SUMMARY OF THE INVENTION

In one general aspect there is provided a method for preparing a multi-layered osmotic tablet having a push end and a dispensing end using rapid and accurate determination of the orientation of tablets.

In another general aspect there is provided a method for preparing a multi-layered osmotic tablet having a push end and a dispensing end; wherein both the ends have different degree of curvature.

In another general aspect there is provided a method for preparing a multi-layered osmotic cylindrical tablet having a push end and a dispensing end; wherein both the ends have different angles of the curved surfaces.

In another general aspect there is provided a method for preparing a multi-layered osmotic tablet having a push end and a dispensing end for laser drilling of a delivery port in said dispensing end, the method comprising the steps of:
detecting the orientation of the tablet by detecting the angle of the curved surface on a side of the tablet corresponding to one or another formulation layer depending on the formulation orientation of the tablet, wherein both the ends have different degrees of curvature;
conveying signal to laser gun regarding orientation of dispensing end;
passing the tablet through a conveyor belt; and
drilling the desired surface using a laser beam.

In another general aspect there is provided a method for determining orientation of the multi-layered osmotic tablet on the basis of the angle of curved surface using an image recognition system.

In another general aspect there is provided a method for rapid and accurate laser drilling on the desired surface of the osmotic tablet without removing improperly oriented tablets before laser drilling.

In another general aspect a multi-layered osmotic tablet comprises methylphenidate or a salt thereof.

In another general aspect a multi-layered osmotic tablet comprises paliperidone or a salt thereof.

In another general aspect there is provided a method for preparing a multi-layered osmotic tablet having a push end and a dispensing end; wherein the dispensing end has higher degree of curvature.

The details of one or more embodiments of the present invention are set forth in the description below, Other features, objects and advantages of the invention will be apparent from the description.

### BRIEF DESCRIPTION OF THE DRAWING

FIG. 1 is a longitudinal sectional view of an asymmetric multi-layered osmotic tablet according to the present invention, wherein, the angle formed between the outer curved surface of the drug-containing layer (i.e., the upper surface) and the lateral surface is θ₁; and the angle formed between the outer curved surface of the push layer (i.e., the lower surface) and the lateral surface is θ₂; the vertical distance from the central vertex of the outer curved surface of the drug-containing layer to the plane formed by the intersection line between the outer curved surface of the drug-containing layer and the lateral surface is L1; and the vertical distance from the central vertex of the outer curved surface of the push layer to the plane formed by the intersection line between the outer curved surface of the push layer and the lateral surface is L2. In the figure, when θ₁=θ₂, the tablet is symmetric multi-layered tablet; when θ₁>θ₂ or θ₁<θ₂, the tablet is asymmetric multi-layered tablet.

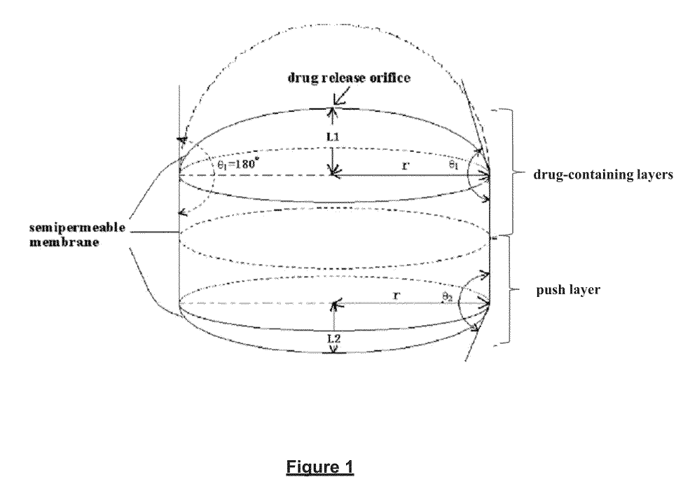

### DETAILED DESCRIPTION OF THE INVENTION

We have surprisingly found that determination of the formulation orientation of multi-layered osmotic capsule-shaped tablets is accurately and efficiently accomplished by a method according to the present invention. The present invention involves detecting the formulation orientation of the tablet by detecting the angle of the curved surface on a side of the tablet corresponding to one or another formulation layer depending on the formulation orientation of the tablet, wherein both the ends have different degrees of curvature; determining the formulation orientation of the tablet on the basis of the degree of curvature; passing the tablet through a conveyor belt; and drilling the desired surface using a laser beam without removing improperly oriented tablets.

The inventors have developed a method for determining orientation of capsule shaped multi-layered tablets using an image recognition system and drilling the desired curved surface without removing improperly oriented tablets.

As per the invention, there is no need to use different coloring agents in the multi-layered osmotic composition for differentiation between a drug-containing compartment and a push compartment. Thus, the composition as per the invention is devoid of any iron content which might be present in the coloring agents and which might be hazardous to health. The multilayered osmotic tablet as described herein may include bi-layered or tri-layered osmotic tablets.

The phrase “different angles of the curved surfaces” is employed herein to refer to θ₁ is not as same as that of θ₂ and the tablet is asymmetric tri-layer capsule shaped tablet.

In one embodiment, there is provided a method for determining orientation of the multi-layered osmotic tablet on the basis of the angle of curved surface using an image recognition system.

In another embodiment, there is provided a method for determining orientation of an asymmetric multi-layered osmotic capsule shaped tablet on the basis of difference in degree of curvature using an image recognition system.

In another embodiment, there is provided a method for preparing a multi-layered osmotic tablet having a push end and a dispensing end for laser drilling of a delivery port in said dispensing end, the method comprising the steps of:
detecting the orientation of the tablet by detecting the angle of the curved surface on a side of the tablet corresponding to one or another formulation layer depending on the formulation orientation of the tablet, wherein both the ends have different degrees of curvature;
determining the formulation orientation of the tablet on the basis of the angle of curved surface;
conveying signal to laser gun regarding orientation of dispensing end;
passing the tablet through a conveyor belt; and
drilling the desired surface using a laser beam.

Here, the image recognition system is based on, but not limited to the theory of light reflectance, refraction, diffraction system or a camera based system capable of capturing the difference in curvature of upper and lower surface of the asymmetric multi-layered osmotic tablet.

In another embodiment, there is provided a method for laser drilling the tablets without removing improperly oriented tablets wherein laser drilling station is present on the both sides of the conveyor belt.

In another embodiment, there is provided a method for preparing a multi-layered osmotic tablet having a push end and a dispensing end for laser drilling of a delivery port in said dispensing end, wherein the dispensing end having higher degree of curvature as compared to that at the push end.

In another embodiment, there is provided a method for determining orientation of the asymmetric multi-layered osmotic tablet on the basis of the angle of curved surface, wherein the angle θ₁ formed by the outer curved surface of the drug containing layer and the lateral surface is in between 120° to 180° and the angle θ₂ formed by the outer curved surface of the push layer and the lateral surface is in between 95° to 150°.

In another embodiment, there is provided a method for determining orientation of the asymmetric multi-layered osmotic tablet on the basis of the angle of curved surface, wherein the angle θ₁ formed by the outer curved surface of the drug containing layer and the lateral surface is in between 95° to 150° and the angle 09 formed by the outer curved surface of the push layer and the lateral surface is in between 120° to 180°.

In another embodiment, there is provided a method for determining orientation of the asymmetric multi-layered osmotic tablet on the basis of the angle of curved surface, wherein the laser beam is applied automatically on the surface having higher or lower degree of curvature.

The osmotic controlled release tablet according to the present invention comprises structurally a capsule shaped tablet core and a semipermeable film coated on the tablet core. The tablet core is composed of a drug and one or more penetration-enhancing agents, fillers, pushing agents, cosolvents, lubricants, adhesives, plasticizers, pore-forming agents, wetting agents or other components.

The drugs useful in the preparations according to the present invention are not limited and selected from for example the group consisting of prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, topiramate, paliperidone, oxybutynin, methyl phenidate, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17-varies hydroxyprogesterone acetate, 19-nor-progesterone, norgestrel, norethindrone, norethisterone, norethiederone, progesterone, norgesterone, norethynodrel, aspirin, acetaminophen, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, capropril, mando, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat, captopril, ramipril, famotidine, nizatidine, sucralfate, etintidine, tetratolol, minoxidil, chlordiazepoxide, diazepam, amitriptyline, imipramine, and terazosine HCl di-hydrate. Further examples are proteins and peptides which include, but are not limited to, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, prolactin, somatostatin, lypressin, pancreozymin, luteinizing hormone, LHRH, LHRH agonists and antagonists, leuprolide, interferons, interleukins, growth hormones such as human growth hormone, bovine growth hormone and porcine growth hormone, fertility inhibitors such as the prostaglandins, fertility promoters, growth factors, coagulation factors, human pancreas hormone releasing factor, analogs and derivatives of these compounds, and pharmaceutically acceptable salts of these compounds or their analogs or derivatives, and various combinations of these compounds, and various combinations of these compounds with various pharmaceutically acceptable salts of the above compounds.

Suitable penetration-enhancing agents include, but are not limited to sucrose, sorbitol, mannitol, glucose, lactose, fructose, sodium chloride, potassium chloride, magnesium sulfate, potassium sulfate, sodium sulfate or a combination thereof.

Suitable fillers include, but are not limited to mannitol, lactose, microcrystalline cellulose, sucrose, sodium chloride, starch, cellulose, dextrin, pre-gelatinized starch, calcium hydrogen phosphate, polyvinyl pyrrolidone, hydroxypropyl methyl cellulose, carboxymethyl cellulose and sodium salt thereof, methyl cellulose, ethyl cellulose or a combination thereof.

Suitable pushing agents include, but are not limited to pharmaceutically acceptable expansible materials such as methyl cellulose, hydroxypropyl cellulose, hydroxypropyl methyl cellulose, polyoxyethylene, carbomer, sodium carboxymethyl starch, carboxymethyl cellulose and sodium salt thereof or cross-linked carboxymethyl cellulose sodium or a combination thereof.

Suitable cosolvents include, but are not limited to sodium dodecyl sulfate, poloxamer, polyethylene glycol, povidone, polyethylene glycol 15 hydroxystearate, tween 80, hydroxypropyl β-cyclodextrin, polyoxyethylene castor oil, polyoxyethylene hydrogenated castor oil, lecithin or a combination thereof.

Suitable lubricants include, but are not limited to magnesium stearate, calcium stearate, zinc stearate, glycerol monostearate, sodium stearyl fumarate, polyoxyethylene monostearate, sucrose monolaurate, sodium lauryl sulfate, magnesium lauryl sulfate, magnesium dodecyl sulfate, talcum powder or a combination thereof.

Suitable adhesives include, but are not limited to polyethylene pyrrolidone, hydroxypropyl methyl cellulose, hydroxypropyl cellulose, carboxymethyl cellulose and sodium salt thereof, methyl cellulose, ethyl cellulose or povidone or a combination thereof.

Suitable wetting agents include, but are not limited to water, anhydrous ethanol, ethanol-water solution at various concentrations.

Suitable plasticizers include, but are not limited to glycerol, propylene glycol, polyethylene glycol, triethyl citrate, dibutyl sebacate, phthalates, polyethylene glycol or a combination thereof.

Suitable pore-forming agents include, but are not limited to hydroxypropyl cellulose, hydroxypropyl methylcellulose, methyl cellulose, glycerol, propylene glycol, polyethylene glycol, sucrose, mannitol, lactose, sodium chloride or a combination thereof.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLE 1

An Asymmetric Multi-Layered Osmotic Tablet of Methylphenidate Hydrochloride

TABLE 1

| | | |
|---|---|---|
| | | Quantity |
| Sr. No. | Ingredients | (mg/tablet) |

First drug containing layer

| | | |
|---|---|---|
| 1 | Methylphenidate hydrochloride | 3.76 |
| 2 | Polyethylene oxide | 33.48 |
| 3 | Polyvinylpyrrolidone | 2.00 |
| 4 | Succinic acid | 0.54 |
| 5 | Stearic acid | 0.20 |
| 6 | Butylated hydroxyl toluene | 0.02 |

| | |
|---|---|
| total quantity of first layer | 40.00 |

Second drug containing layer

| | | |
|---|---|---|
| 7 | Methylphenidate hydrochloride | 10.24 |
| 8 | Polyethylene oxide | 59.14 |
| 9 | Polyvinylpyrrolidone | 3.75 |
| 10 | Succinic acid | 1.46 |
| 11 | Stearic acid | 0.37 |
| 12 | Butylated hydroxyl toluene | 0.04 |

| | |
|---|---|
| total quantity of second layer | 75.00 |

Third layer (Push layer)

| | | |
|---|---|---|
| 13 | Polyethylene oxide | 66.72 |
| 14 | Sodium chloride | 18.32 |
| 15 | Polyvinylpyrrolidone | 4.50 |
| 16 | Stearic acid | 0.23 |
| 17 | Butylated hydroxyl toluene | 0.23 |

| | |
|---|---|
| total quantity of third layer | 90.00 |

Semipermeable membrane

| | | |
|---|---|---|
| 18 | Cellulose acetate | 21.33 |
| 19 | Poloxamer | 4.37 |

Immediate release coat

| | | |
|---|---|---|
| 20 | Methylphenidate hydrochloride | 4.00 |
| 21 | Hypromellose | 9.33 |

Film Coating

| | | |
|---|---|---|
| 22 | Opadry | 16.90 |
| 23 | Water | q.s. |

| | |
|---|---|
| TOTAL | 260.93 |

Process:

1. Preparation of a Tablet Core Coated with Semipermeable Membrane

Each of the first component layer, second component layer and third push layer were separately prepared into granulated compositions in a fluid bed granulator or rapid mixer granulator. The granulated compositions were then compressed sequentially on a rotary tablet press to produce the asymmetric multi-layered tablet cores. For each dosage form, 40 mg of the first component layer granulation and 75 mg of the second component layer granulation were first sequentially filled and tamped at required compression force into the die. Then, 90 mg of the third push layer granulation to the die was added to the die and the final compression was performed at required compression force. Here, tablet cores were prepared in such a way that both end having different angles of the curved surface. For preparing semipermeable membrane 83% by weight cellulose acetate and 17% by weight copolymer of ethylene and propylene oxide were dissolved in a blend of 99.5% acetone and 0.5% water to form a 5% solution. In a pan coater, the solution was then sprayed onto the asymmetric multi-layered tablet cores.

2. Detection of Angle of the Curved Surfaces and Laser Drilling

Tablets coated with semipermeable membrane were then subjected to an image recognition system which detects angle of the curved surfaces. On the basis of angle detected by the image recognition system, the proper site for laser drilling was determined automatically and after determining orientation, coated tablets were passed through conveyor belt which was surrounded by laser drilling station from both sides. Based on orientation of the tablet, either top or bottom located laser gun executed laser firing on the dispensing ends.

3. Immediate Release Coating onto Laser Drilled Tablets

The drug overcoat for providing an immediate-release initial dose of drug contained approximately 30% by weight methylphenidate hydrochloride, approximately 70% by weight hypromellose (HPMC). An aqueous coating solution was prepared by dissolving and mixing the ingredients in water to form a solution with 10% solids composition. In a pan coater, the solution was then sprayed onto the semipermeable membranes of the tri-layer osmotic dosage forms to a weight of about 14.0 mg comprising an immediate-release dose of methylphenidate of about 4 mg.

4. Final Aesthetic Overcoat

The tablets were finally coated with aqueous opadry solution.

### EXAMPLE 2

An Asymmetric Multi-Layered Osmotic Tablet of Paliperidone

TABLE 2

| | | |
|---|---|---|
| | | Quantity |
| Sr. No. | Ingredients | (mg/tablet) |

First drug containing layer

| | | |
|---|---|---|
| 1 | Paliperidone | 2.00 |
| 2 | Polyethylene oxide | 31.73 |
| 3 | Sodium chloride | 7.00 |
| 4 | Hydroxypropyl methylcellulose | 4.00 |
| 5 | Stearic acid | 0.250 |
| 6 | Butylated hydroxyl toluene | 0.025 |
| 7 | Water | q.s. |

Second drug containing layer

| | | |
|---|---|---|
| 8 | Paliperidone | 4.00 |
| 9 | Polyethylene oxide | 41.725 |
| 10 | Hydroxypropyl methylcellulose | 4.00 |
| 11 | Stearic acid | 0.250 |
| 12 | Butylated hydroxyl toluene | 0.025 |
| 13 | Water | q.s. |

Third layer (Push layer)

| | | |
|---|---|---|
| 14 | Polyethylene oxide | 75.00 |
| 15 | Sodium chloride | 20.00 |
| 16 | Polyvinylpyrrolidone | 13.73 |
| 17 | Stearic acid | 0.25 |
| 18 | Butylated hydroxyl toluene | 0.015 |
| 19 | Water | q.s. |

Cellulose acetate coat

| | | |
|---|---|---|
| 20 | Cellulose acetate | 44.55 |
| 21 | Polyethylene glycol | 0.45 |
| 22 | Acetone | q.s. |
| 23 | Water | q.s. |

Film Coating

| | | |
|---|---|---|
| 24 | Opadry | 10.00 |
| 25 | Water | q.s. |

| | |
|---|---|
| TOTAL | 259.00 |

Process:

1. Preparation of a Tablet Core Coated with a Semipermeable Membrane

Each of the first component layer, second component layer and third push layer were separately prepared into granulated compositions in a fluid bed granulator or rapid mixer granulator. The granulated compositions were then compressed sequentially on a rotary tablet press to produce the asymmetric multi-layered tablet cores. Here, tablet cores were prepared in such a way that both the ends have different angles of the curved surface. For preparing semipermeable membrane, cellulose acetate and polyethylene glycol were dissolved in acetone and water to form a clear solution. In a pan coater, the solution was then sprayed onto the tri-layer capsule shaped tablet cores.

2. Detection of Angle of the Curved Surfaces and Laser Drilling

Tablets coated with semipermeable membrane were then subjected to an image recognition system which detects angle of the curved surfaces. On the basis of angle detected by the image recognition system, the proper site for laser drilling was determined automatically and after determining orientation, coated tablets were passed through conveyor belt which was surrounded by laser drilling station from both sides. Based on orientation of the tablet, either top or bottom located laser gun executed laser firing on the dispensing ends.

3. Final Aesthetic Overcoat

The tablets were finally coated with aqueous opadry solution.

1. A method of preparing a multi-layered osmotic tablet having a push end and a dispensing end for laser drilling of a delivery port in the dispensing end, the method comprising the steps of:
detecting an orientation of the tablet by detecting an angle of the curved surface on a side of the tablet corresponding to one or another formulation layer depending on the formulation orientation of the tablet, wherein both the ends have different degrees of curvature;
conveying signal to a laser gun regarding orientation of the dispensing end;
passing the tablet through a conveyor belt; and
drilling the desired surface using a laser beam.

2. The method according to claim 1, wherein the laser gun is present on the both sides of the conveyor belt.

3. The method according to claim 1, wherein the angle of the curved surface is detected by an image recognition system.

4. The method according to claim 1, wherein the multi-layered osmotic tablet comprises methylphenidate or a salt thereof.

5. The method according to claim 1, wherein the multi-layered osmotic tablet comprises paliperidone or a salt thereof.

6. The method according to claim 1, wherein the dispensing end has higher degree of curvature.

7. The method according to claim 1, wherein the tablet comprising an angle formed by the outer curved surface of the drug containing layer and the lateral surface is in between 120° to 180°.

8. The method according to claim 1, wherein the tablet comprising an angle formed by the outer curved surface of the push layer and the lateral surface is in between 95° to 150°.

9. The method according to claim 1, wherein the laser beam is applied automatically on the surface having higher degree angle of the curved surface.
